(19) United States
(12) Patent Application Publication
(10) Pub. No.: US 2010/0299075 A1
(43) Pub. Date: Nov. 25, 2010

(54) SYSTEMS AND METHODS FOR CALCULATING AN AVERAGE ANALYTE CONCENTRATION VALUE

(75) Inventors: Qiong Li, Tappan, NY (US); Scott Pardo, Wesley Hills, NY (US); Stanley A. Telson, White Plains, NY (US)

Correspondence Address: Dugan & Dugan, PC, 245 Saw Mill River Road, Suite 309, Hawthorne, NY, 10532, US

(73) Assignee: Bayer HealthCare LLC, Tarrytown, NY (US)

(21) Appl. No.: 12/777,199

(22) Filed: May 10, 2010

Related U.S. Application Data
(60) Provisional application No. 61/179,477, filed on May 19, 2009.

(51) Int. Cl. G06F 15/00; G01N 33/48

(52) U.S. Cl. 702/19; 702/199

(57) ABSTRACT

Embodiments provide methods and systems wherein analyte concentration readings taken over a first period of time are collected and processed to determine one or more analyte concentration averages. The methods include collecting samples with a measurement system (e.g., a Blood Glucose Meter) over a first period of time, dividing the first period of time into smaller time increments, and calculating an average analyte concentration based on first sub-averages obtained from each of the smaller time increments. Systems for carrying out the analyte concentration averages are described, as are other aspects.

### RELATED APPLICATIONS

The present application claims priority to U.S. Provisional Patent Application No. 61/179,477, filed May 19, 2009, and entitled “SYSTEMS AND METHODS FOR CALCULATING AN AVERAGE ANALYTE CONCENTRATION VALUE” (Attorney Docket No. BHDD-019/L), which is hereby incorporated herein by reference in its entirety for all purposes.

### FIELD OF THE INVENTION

The present invention relates generally to systems and methods for calculating average analyte values.

### BACKGROUND OF THE INVENTION

The quantitative determination of analytes in body fluids may be important in the diagnoses and maintenance of certain physiological conditions. For example, individuals with diabetes frequently check a blood glucose concentration level in their bio-fluids (e.g., blood). The results of such tests may be used to regulate a glucose intake in their diets and/or to determine whether insulin or other medication may be needed.

Diagnostic systems, such as analyte measurement systems, may employ an analyte meter to calculate an analyte concentration level (e.g., a glucose concentration level) in a blood sample taken from an individual, for example. Such analyte meters may operate by measuring an output, such as an electrical current or color change, from a reaction with the analyte contained in the sample. The test concentration readings results may be stored by the analyte meter and may be displayed to the user in simple form. Basic processing systems in the analyte meter may allow the user to access the test results directly from the analyte meter. However, the graphical display capability, because of their small size may be limited. Furthermore, the processing and storage capabilities on many analyte meters may be inadequate for analysis that is more sophisticated.

To manage the test data (e.g., analyte concentration readings) according to more advanced functionalities that go beyond simple meter display and/or storage of test results, a user may wish to download the data onto another processing device. The processing device may be a conventional desktop personal computer (PC), or a hand-held device, such as a cell phone or PDA wherein a separate health data management application may be executed. As part of this enhanced analysis, the health data management application may plot data over a period of time, such a week, two weeks or a month and calculate an average analyte concentration level over that period of time. In particular, certain conventional analysis techniques may utilize a running analyte concentration average, for example. The running average sums all of the analyte readings taken over a period of time and divides that sum by the number of samples. However, the inventors have determined that the running average technique may lead to a biased result and inaccuracies in the displayed average concentrations, especially when readings are not evenly distributed in time.

Accordingly, systems and methods, which may allow improved average analyte calculations and data display may be desirable.

### SUMMARY OF THE INVENTION

According to a first aspect, a method to determine an average analyte value is provided. The method includes measuring a plurality of analyte concentration readings over a time period with an analyte measurement device; selecting a first period of time within the time period; dividing the first period of time into a plurality of smaller time increments each including at least three analyte concentration readings; averaging the analyte concentration readings within the plurality of smaller time increments to calculate first sub-averages; averaging two or more of the first sub-averages to calculate at least one second sub-average; and displaying at least one average selected from the group of: an average based on the first sub-averages, an average based on the at least one second sub-average, and an overall analyte average over the first period of time based on the at least one second sub-average.

In another method aspect, a method to determine an average analyte value is provided. The method includes measuring a plurality of analyte concentration readings with an analyte measurement system over a time period; downloading the plurality of analyte concentration readings to a host device; selecting a first period of time within the time period; dividing the first period of time into a plurality of smaller time increments of substantially equal lengths; averaging the analyte concentration readings for each of the smaller time increments to determine first sub-averages for each of the smaller time increments; averaging the first sub-averages to arrive at a plurality of second sub-averages; averaging the second sub-averages to arrive at at least one third sub-average; and displaying on the host device at least one average selected from the group of: the first sub-averages, the second sub-averages, the at least one third sub-average, and an overall analyte average over the first period of time.

According to another aspect, a system adapted to calculate an average analyte value is provided. The system includes a measurement system adapted to obtain a plurality of analyte concentration readings; a host device adapted to receive at least some of the plurality of analyte concentration readings, the host device including a processor adapted to calculate an analyte concentration average over a selected first period of time wherein a plurality of analyte readings over the first period of time are stored in memory and the processor calculates an analyte concentration average based on dividing the first period of time into a plurality of smaller time increments, each smaller time increment including at least three analyte concentration readings, averaging the analyte concentration readings for each of the smaller time increments to determine a first sub-average for each of the smaller time increments, and averaging at least some of the first sub-averages to arrive at at least one second sub-average.

Still other aspects, features, and advantages of the present invention may be readily apparent from the following detailed description by illustrating a number of exemplary embodiments and implementations, including the best mode contemplated for carrying out the present invention. The present invention may also be capable of other and different embodiments, and its several details may be modified in various respects, all without departing from the spirit and scope of the present invention. Accordingly, the drawings and description are to be regarded as illustrative in nature, and not as restrictive. The invention is to cover all modifications, equivalents, and alternatives falling within the spirit and scope of the invention.

### BRIEF DESCRIPTION OF THE DRAWINGS

FIG. 1 is a graphical illustration of exemplary analyte concentration readings taken over a time period according to the prior art.

FIGS. 2a and 2b illustrate a method of generating an average glucose reading according to the prior art.

FIG. 3 is a graphical illustration of an exemplary system adapted to generate and display an average glucose reading according to embodiments of the present invention.

FIG. 4 illustrates a block diagram of an exemplary method for calculating an average analyte value according to embodiments of the present invention.

FIG. 5 illustrates a block diagram of another exemplary method for calculating an average analyte value according to embodiments of the present invention.

FIGS. 6A-6C are graphical illustrations displaying analyte concentration readings according to embodiments of the present invention.

FIG. 7 is a flowchart illustrating a method according to embodiments of the present invention.

### DETAILED DESCRIPTION

In view of the foregoing difficulties, there is a need for systems and methods, which may accurately calculate an average analyte value. To address this need, embodiments according to aspects of the present invention, in a first aspect, provide a method for calculating one or more analyte average values.

In particular, methods according to embodiments of the invention may include providing a plurality of analyte concentration readings over a first period of time from a measurement system, such as an analyte meter. Examples of analyte measurement systems include analyte meters such as Blood Glucose Meters (BGMs), Continuous Glucose Meters (CGMs), and Pump/CGM combinations. According to the method, the first period of time is divided into a plurality of smaller time increments, such as 15 minutes, a half hour, an hour, 24 hours, a week, etc. A first sub-average (e.g., an arithmetic average) may be calculated for the concentration readings within each of the smaller time increments. Each smaller increment may include greater than three analyte concentration readings. Thus, a plurality of first sub-averages may be obtained over the selected first period of time. For each of these smaller time increments, some readings may be missing or may be determined to be improper readings based upon filtering and/or a checking routine, for example. In this case, such missing and improper readings may be discarded (ignored) and are not included in the average calculations for the smaller time increments. Various additional sub-averages may be calculated based upon the first sub-averages obtained for each smaller time increment. For example, second sub-averages and third sub-averages may be calculated. Further, an overall analyte average over the first period of time may be calculated.

The overall analyte average calculated by the method may then be displayed for the first period of time. Any useful period may be chosen for display, such as a week, a day, an hour, etc. In some embodiments, the first period of time may be selected by a user and sub-averages for each suitable time sub-period within the first period of time may be calculated and displayed to the user. For example, a user may select a period between Mar. 19, 2009 and Apr. 26, 2009, and the method may calculate for display a weekly overall analyte average. The weekly overall average may be the average of all the weekly sub-averages within the first period of time. Each weekly sub-average may be based upon an average of all 24-hour sub-averages within that week. Furthermore, the method may calculate for display a 24-hour overall analyte average, which may be the average of all 24-hour analyte sub-averages within the selected first period of time. Likewise, the method may calculate for display an hourly overall analyte average, which may be the average of all hourly analyte sub-averages within the selected first period of time. Overall averages for other suitable sub-periods of time may be calculated and displayed. For example, in further embodiments, daytime, nighttime and meal overall sub-averages may be calculated and displayed. Additionally, statistical displays of the individual sub-averages over the first period of time for one or more sub-periods may be displayed such that the extent of outliers may be determined and/or examined. Furthermore, event markers for events may be included such as meal markers, exercise markers, insulin markers, and the like. Analyte concentration sub-averages may be calculated for display for sub-periods of time adjacent to any such event. Thus, the present invention provides a method for providing overall analyte concentration averages, which may take into account the time varying nature of the readings, missing data points, and/or large gaps in the data.

Accordingly, the present invention has utility for providing an analyte concentration average over any particular period of time that may be relatively less affected by missing readings, improper readings, and/or data gaps. Moreover, the present invention may overcome inaccuracies that may accrue via the use of time invariant averaging methods. Furthermore, the method may calculate sub-averages for any suitable sub-periods of time. These sub-averages may also be less sensitive to time varying (e.g., unevenly spaced, missing, and/or large gaps in the analyte concentration readings). The embodiments described herein are advantageous to those individuals who are actively involved in monitoring and/or recording measurements of their health information, such as test data concerning blood analyte concentrations (e.g., glucose concentrations).

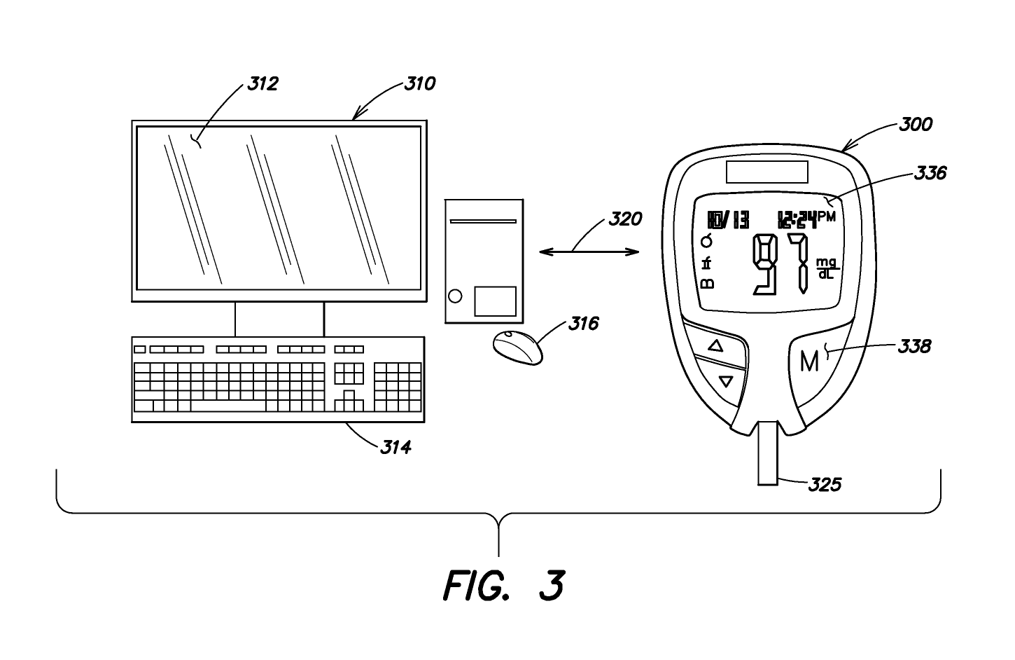

These and other embodiments of the methods and systems of the present invention are described below with reference to FIGS. 3-7.

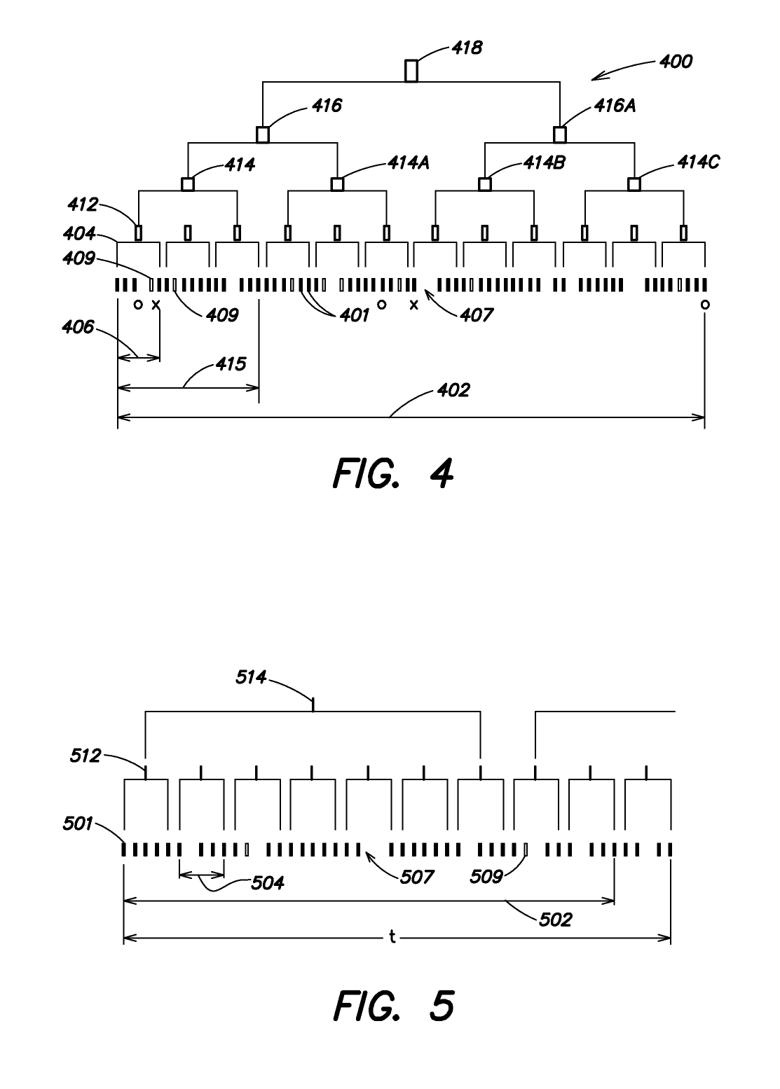

Referring to FIGS. 3-4, a nonlimiting example of a system and method according to embodiments of the invention is generally illustrated. In particular, the method 400 may include a plurality of analyte concentration measurements (readings) 401 taken on a measurement device 300. The measurements may take place in even increments (e.g., every minute, every few minutes, every hour, or the like) such as when an automated measurement device takes the reading (e.g., a CGM). In other embodiments, the analyte readings may be taken in nonequal time increments, such as when a user determines the frequency of analyte readings by using a measurement device (e.g., the analyte meter shown in FIG. 3). Thus, the analyte readings 401 may be blood analyte readings supplied from a measurement device 300 such as a BGM, a CGM, a Pump/CGM combination, or the like. The analyte readings may be processed in the measurement device 300 (e.g., by the BGM, CGM, or Pump/CGM combination), and/or may be downloaded from the measurement device 300 onto a host device 310.

Both the host device 310 and the measurement device 300 may include a suitable communication connection 320 enabling data communication between the measurement device 300 and the host device 310. The communication connection may take on any form, such as a wireless connection, a USB connection, download from a memory article such as a disc, flash drive, or the like. In some embodiments, a calculation of an analyte average according to the method of the invention may take place within the processor of the measurement device 300. The averages and other data may be displayed on the measurement device 300 and/or on the host device 310, for example. In other embodiments, the measurements may be obtained via the measurement device 300 and downloaded to the host device 310 wherein the calculations are carried out by the processor of the host device 310 and may be displayed on a display 312 of the host device 310.

The host device 310 may be selected from a variety of processing devices, such as desktop or laptop personal computers (PCs), hand-held or pocket personal computers (HPCs), compatible personal digital assistants (PDAs), and smart cellular phones, for example. Other types of smart devices, i.e., those including a digital processor, memory and a suitable graphical display, also may be used. To operate, the host device 310 may employ a variety of operating systems and/or configurations. For example, if the host device 310 is a desktop or laptop personal computer, the operating system may be a version of MICROSOFT® WINDOWS®. Alternatively, if the host device 310 is a PDA, the operating system may correspond with those of PALM® hand-fields from Palm, Inc., or BLACKBERRY® devices from Research in Motion Limited. Any suitable operating system may be used on the host device 310 for calculations of analyte averages according to the method and/or for display of the analyte averages.

The host device 310 may include a conventional digital processor that is adapted to and capable of receiving data in digital form and executing any number of programmed instructions. In addition, the host device 310 may be typically operated with a user interface, which may include the display 312 (e.g., an LCD display) and/or a keyboard 314, a mouse 316, or other input device, which may be external to, or integrated with, other components of the host device 310.

The host device 310 may also include a memory, such as a Random Access Memory (RAM) (including EDO, SDRAM, DDR SDRAM, SIMM, DIMM) and/or nonvolatile memory such as Read-Only Memory (ROM) including Programmable Read-Only Memory (PROM), and Electrically Erasable Programmable Read-Only Memory (EEPROM). The memory may also include storage technologies, such as one or more storage devices such as a hard drive, disk, CD, etc. It is contemplated the memory may be configured to include any combination and form of RAM, ROM, and/or storage technologies. The memory may be provided as a separate unit or incorporated as part of the digital processor.

In some embodiments, the memory may store software associated with a health data management system (hereinafter “health data management software”). The health data management software may be a collection of programs or computer codes that receive and process measured analyte concentration readings and/or other associated health data (e.g., date, time, meal markers, insulin markers, exercise markers, etc.) and/or other user input. The health data management software may further process and/or display or plot the readings, health data, and/or related information in a manner desired by the user. This collective measured health information may be used by, for example, a user, home care provider (HCP), and/or a physician.

As discussed above, the measured health information may include analyte concentration readings (analyte test data) and related information from a testing of analyte concentrations over a time period. At least some of the test data may be generated by, and downloaded from, the measurement device 300 (e.g., an analyte meter). For example, the test data may include a concentration of glucose and/or other analyte concentrations in a person's blood or other bio-fluid as well as related information (time, date, meal markers, insulin markers, exercise markers, and the like). Advantageously, the health data management software may provide advanced displays and data processing that may be desired by a user who may test multiple times a day (e.g., from about six to about ten times a day). For example, the health data management software may include a product similar to WinGLUCOFACTS™ Diabetes Management Software available from Bayer HealthCare LLC (Tarrytown, N.Y.). The health data management software may:
receive and store analyte readings from an analyte measurement device,
receive and store other related testing information such as test times, dates, meal markers, insulin markers, exercise markers,
track analyte readings in an electronic logbook,
calculate analyte averages of the analyte readings in accordance with aspects of the invention and provide statistical analysis of analyte readings,
summarize and provide feedback on the analyte readings,
provide a customizable graphical user interface,
display user-friendly charts and graphs of the analyte readings,
track analyte readings against user-specific target ranges,
provide predictive analysis, and/or
send analyte readings and averages to healthcare professionals.

It should be recognized that although some embodiments according to the present invention may be readily adapted to calculate averages (sub-averages and overall averages of a first period of time) of analyte readings, other uses of the invention are also contemplated. In other embodiments, any slow-changing test data may be received from a measurement device that measures and/or records health data, and average concentration calculations according to the methods described herein may be made thereon.

In some embodiments, the measurement device 300 may include a suitable digital processor and memory for storage of analyte test data and related information, carrying out measurements and calculations of analyte concentration levels, and carrying out the processing of the analyte readings, for example. The digital processor and memory may include any suitable digital processor, microprocessor, and memory such as those described above. As illustrated, the measurement system 300 may receive and engage a test sensor 325 (sometimes referred to as a “test strip”). The measurement device 300 may include a port for receiving a test sensor 325. The measurement device 300 is adapted to measure a concentration of an analyte for the sample collected on the test sensor 325. The actual calculation of the analyte concentration readings from a reaction measured by the analyte meter 300 and the procedure for testing the sample may be accomplished by the digital processor, which may execute programmed instructions according to a conventional measurement algorithm contained in software. Analyte readings processed by the digital processor may be stored in memory. Thus, a plurality of analyte concentration readings may be taken and stored over a time period.

The measurement system 300 may include a user interface, which may include a suitable display 336 such as a liquid-crystal display for displaying analyte test data and results (e.g. analyte concentration averages) to the user, and a user input 338, such as one or more push buttons, a scroll wheel, touch screens, and/or any combination thereof, for providing user input.

In some embodiments, the memory and processing capability of the measurement device 300 may be sufficient so that software for performing advanced analysis of the analyte concentration readings may be stored and may be operative in the measurement device 300. For example, in some embodiments, in addition to the conventional software routines for calculating and displaying individual analyte concentration readings, the data management software adapted to perform advanced analysis and display of the analyte concentration readings may be stored in the memory resident on the measurement device 300, as well. The memory may include the types of memory mentioned above for the host device 310, but may also include a flash memory device, such as a universal serial bus (USB) flash drive, or a memory card. USB flash drives are also known as thumb drives, handy drives, flash sticks, or jump drives. Memory cards may have a variety of formats, including PC Card (PCMCIA), CompactFlash (CF), SmartMedia (SM/SMC), Memory Stick (MS), Multimedia Card (MMC), Secure Digital Card (SD), xD-Picture Card (xD), Intelligent Stick (iStick), ExpressCard, or some variation thereof. Flash memory devices may employ nonvolatile memory so that the software stored therein may be retained in the memory even when the memory receives no power. It is also contemplated that the memory may employ other storage media, such as floppy disk or optical disc (CD, DVD, Blu-ray disc).

In some embodiments, the memory in the measurement device 300 may include execute-in-place (XIP) memory, such as NOR (NOR digital logic gate) flash memory, so that the health data management software which may be stored locally in the memory and may be executed directly without the need to copy the data into RAM on the host device 310. Accordingly, some embodiments may secure the data by ensuring that all data may be stored in memory and processed by the processor running locally within the measurement system 300 in the user's possession. In this fashion, no data may be transferred to the host device 310 so that other individuals might be able to access. Thus, a user may use, for example, a public computer as the host device 310 to interface with the measurement system 300 and provide enhanced displays the data, and yet no data will remain on the host device 310 for others to see.

According to some embodiments, a combined system is provided including the measurement device 300 adapted to generate a plurality of analyte concentration readings and being adapted for communication with a host device 310. A communication connection 320 may be adapted to allow downloading to the host device 310 of the plurality of analyte concentration readings taken over a time period by the measurement device 300. In some embodiments, the communication connection 320 may include a USB connection (e.g., a USB cable or connector) connected between the measurement device 300 and the host device 310.

Referring again to FIG. 3, and as stated before, the measurement device 300 may include a test sensor 325. The test sensor 325 may be configured to receive a fluid sample that is analyzed using the measurement device 300. Analytes that may be analyzed may include glucose, lipid profiles (e.g., cholesterol, triglycerides, LDL and HDL), microalbumin, hemoglobin A_{1C}, fructose, lactate, keytones or bilirubin. It is contemplated that analyte data may be determined (e.g., analyte concentration levels) by the measurement device 300, and such analyte concentration readings may be stored locally in memory and communicated periodically to a host device 310. The analytes may be in, for example, a whole blood sample, a blood serum sample, a blood plasma sample, other body fluids like ISF (interstitial fluid) and urine.

The test sensor 325 may include a fluid-receiving area for receiving a sample of body fluid. For example, a user may employ a conventional lancet or a lancing device to pierce a finger or other area of the body to produce the blood sample at the skin surface. The user may then collect this blood sample and place the sample in contact with the fluid-receiving area of the test sensor 325. The fluid-receiving area may contain a reagent that is adapted to react with the sample to indicate information related to an analyte contained in the sample, such as analyte concentration. Such reagents are well known in the art.

The test sensor 325 may be an electrochemical test sensor or a photochromic test sensor. An electrochemical test sensor may typically include a plurality of electrodes and a fluid-receiving area that contains the reagent. Upon contact with the analyte of interest (e.g., glucose) in a fluid sample (e.g., blood) an electrical current may be produced which may be proportional to an analyte concentration level in the sample. The reagent may contain an enzyme such as, for example, glucose oxidase. However, it is contemplated that other reagents may be used to react with the analyte, which may be desired to be measured. In general, the reagent is selected to react with the desired analyte or analytes to be tested to assist in determining an analyte concentration of a fluid sample. If the concentration of another analyte is to be determined, an appropriate enzyme is selected to react with the analyte.

Alternatively, the test sensor 325 may be a photochromic test sensor. Photochromic test sensors may use techniques such as, for example, transmission spectroscopy, diffuse reflectance, or fluorescence spectroscopy for measuring an analyte concentration. An indicator reagent and an analyte in a sample of body fluid may be reacted to produce a chromatic reaction, wherein the reaction between the reagent and analyte causes a color change. The degree of color change is indicative of the analyte concentration in the body fluid. The color change may be evaluated to measure the absorbance level of the transmitted light.

Some commercially-available test sensors 325 that may be used by the embodiments described herein include those that are available commercially from Bayer HealthCare LLC (Tarrytown, N.Y.). These test sensors 325 may include, but are not limited to, those used in the Ascensia® CONTOUR® blood glucose monitoring system, the Ascensia® BREEZE® and BREEZE®2 blood glucose monitoring system, and the Ascensia® Elite® and Elite® XL blood glucose monitoring system. It is contemplated that other test sensors, in addition to the ones listed above, may be incorporated into the methods and systems of the present invention. In accordance with embodiments of the invention, once the analyte concentration readings over a time period are obtained, processing of the analyte test data may be undertaken to calculate various average analyte readings.

FIG. 4 illustrates, according to embodiments of the invention, a method of calculating an overall average analyte value over a first period of time 402. The first period of time 402 may be selectable, such as by a user. The method may also calculate sub-averages for sub-periods occurring within the first period of time 402. In some embodiments, the method 400 includes, providing a plurality of analyte concentration readings 401 (shown as tick marks) over the first period of time 402. The first period of time 402 may be a subset of available data taken over a time period. For example, the plurality of analyte concentration readings 401 may be generated by using an analyte measurement device 300 (FIG. 3), such as an analyte meter, BGM, CGM, pump/CGM combination, or the like. Thus, the analyte concentration readings 401 may be generated responsively to actions taken by a user or generated automatically according to a preset timing interval or plan. Accordingly, the plurality of analyte concentration readings 401 may be taken in equal or nonequal time increments. In some embodiments, the analyte concentration readings 401 may be stored in memory of the measurement device 300. The user may also download the analyte concentration readings 401 to a host device 310. Accordingly, in some embodiments, the readings may be stored in a host device 310. The readings 401 may be downloaded all at once, or in smaller groups, and may be downloaded within a date range or time range selected by the user, for example.

Once the first period of time 402 is defined, such as by a length of time selected by the software based on the amount of available data, a largest even timeframe for which data is available (e.g., day, week, month, etc.), or a user selecting a certain date range to be analyzed (e.g., Jan. 1, 2009 through Jan. 7, 2009), the first period of time 402 may be further divided according to aspects of the method. For example, the first period of time 402 may be divided into a plurality of smaller time increments 404. These smaller time increments 404 may be equal or unequal in duration. The plurality of smaller time increments 404 may each include between 3 and 100 analyte readings, or even between 3 and 60 analyte readings 401, for example. In some embodiments, even more analyte readings may be included in each time increment 404. As shown in FIG. 4, the first period of time 402 may be divided into twelve equal length smaller time increments 404 having time intervals 406. More or less number (m) of time increments 404 over the first period of time may be used. The number (m) of time increments 404 may be selected based on the amount of total data available, for example. In other embodiments, the number (m) of time increments may be based on providing a suitable accuracy level in the averages generated by the method. For example, if a relatively high accuracy level in the average is desired, then the number (m) may be selected so that m>5. Likewise, the number (k) of analyte concentration readings 401 used in each time increment 404 may be selected to be k>5. For cases where a lesser relative accuracy may be tolerated, lower values for m and k may be used. Moreover, if greater relative accuracy is desired, then higher values of m and k may be used. In some embodiments, m and k are selected so that m>5 and k>5. Moreover, if the data is highly correlated, then m and k selected may be made to be lower.

Within each individual time increment 404, there may be one or more missing data points 407, for example. For instance, the measurement device 300 and/or sensor 325 and/or an individual test may have been flawed or improperly carried out and may have resulted in a missing data point 407. A missing data point 407 is one where, for whatever reason, there is a time and date stamp, but no data or a zero reading. Furthermore, there may be one or more improper data points 409 (shown as hollow tick marks) which, although obtained, are determined to be improper in magnitude. For example, a filter or checking routine may be used to check a magnitude of all analyte readings taken and discard/disregard the improper data points 409. In some embodiments, a simple filter with preset thresholds may discard a reading, which is too high or too low (above or below the preset threshold values). In some embodiments, the checking routine may determine, based on a characteristic of one or more previous readings, whether the reading is an improper data point 409. For example, a slope (analyte reading rate change) may be determined for the previous few analyte concentration readings. If the rate of change of the analyte reading is outside of a preset slope threshold set in the checking routine thereby indicating that the reading is physiologically impossible over that period of time, then the reading may be determined to be an improper data point and discarded. In other embodiments, a running average may be calculated, and the next data point compared to that running average value to determine if the data point is an improper data point 409. For example, if the average value change is too large (above a preset threshold), then the analyte value may be deemed to be an improper data point and may be discarded. In other embodiments, if an absolute difference between a value of a previously-obtained analyte reading and the improper data point 409 is above a threshold, then the analyte reading may be discarded as being an improper data point 409. Other methods for determining improper data points 409 to be discarded may be used. The thresholds described above may be selected based upon physiological factors, which indicate that such an analyte concentration reading could not exist for the particular analyte being measured. For example, the threshold may be set based upon a hypothetically impossible maximum and minimum change of analyte concentration over time. With each improper data point 409 and each missing data point 407 being thrown out, a calculation of a first sub-average 412 for each increment 404 may be obtained. The first sub-average 412 for each may be an arithmetic average of the remaining data points in each respective increment 404.

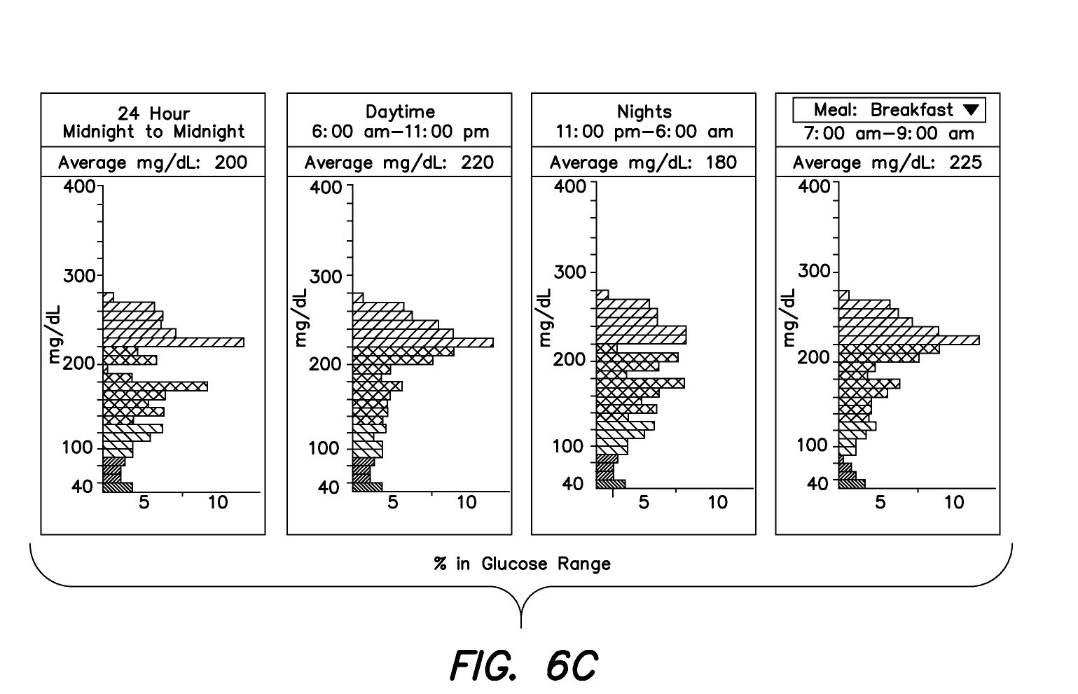

From the first sub-average 412, and at least one other first sub-average from one or more adjacent increments 404, a second sub-average 414 may be calculated. The second sub-average 414 may be obtained by averaging (e.g., an arithmetic average) at least two or more first sub-averages 412. In the depicted embodiment, the second sub-average 414 may be based on an arithmetic average of three adjacent increments 404 resulting in a sub-average over the sub-period 415. The second sub-average 414 may be an average of less than all of the first sub-averages 412 (as shown in FIG. 4), or may be based on all of the available data in the selected first period of time 402. An overall first sub-average may be obtained by averaging all the first sub-averages 412 for all time increments 404 over the first period of time 402. Thus, the overall first sub-average may represent the calculated average for a shortest sub-period within the selected first period of time (e.g., an hourly average). A plot 600 or display of the first sub-averages may be provided as shown in FIG. 6A. Within the plot, an overall first sub-average over the selected first period of time 402 may be displayed, such as to the user, in numerical form in block 601. In some embodiments, the plot 600 may include a graphical distribution 602 (such as a bar chart) of the first sub-averages for the increments 404. Other types of graphical displays may be used (such as pie charts, line charts, etc.). The respective bars may be shaded or colored to reflect various conditions (e.g., in range, over range, under range). For example, in range may be shaded or colored in one way, whereas other groups of bars may be shaded differently. The different colors/shading may be indicative of various conditions, for example.

According to the method, additional second sub-averages may be calculated for groups of at two or more increments 404. For example, additional second sub-averages 414A-414C may be obtained via averaging additional groups of first sub-averages from consecutive groups of increments 404. At least two, such as adjacent ones, of each of these second sub-averages (e.g., 414, 414A) may be averaged (e.g., arithmetically) to obtain a third sub-average 416. Similarly, other (e.g., adjacent) second sub-averages 414B, 414C may be averaged to obtain another third sub-average 416A over another sub-period. Thus, third sub-averages (e.g., 416, 416A) may be calculated based on the second sub-averages (414-414C). In some embodiments, all the second sub-averages may be averaged to provide an overall second sub-average over the first period of time 402. This overall second sub-average may represent a sub-average over a second sub-period, such as 3 hours, 6 hours, 12 hours, 24 hours, etc. Other time periods may be selected. The overall second sub-average may be displayed in a plot such as FIG. 6B, and may include a bar chart showing a distribution of the second sub-averages. This plot illustrates the distribution of the second sub-averages of the averages of the three hourly first sub-averages. However, it should be recognized that the method may utilize averages of any convenient number of time increments 404. For example, by averaging six time increments 404, six-hour averages may be calculated and displayed. Further, the time increments may be selected based on achieving a certain confidence level.

The third sub-averages may be similarly plotted. By averaging the second sub-averages, a third sub-average may be obtained which represents averages of some multiple of the timeframe for the second sub-averages. As shown in FIG. 4, the third sub-average 416 may represent averages over a six-hour period, for example. An overall third sub-average, which is an average of all the third sub-averages, may be displayed in a plot similar to FIG. 6B, and may include a bar chart showing a distribution of the third sub-averages.

Finally, an overall analyte average 418 over the first period of time 402 may be obtained by averaging all the third sub-averages 416, 416A. The overall averages may uniquely account for the time varying nature that may exist in the analyte readings, as well as for missing data points 407 and/or improper data points 409. If enough data is available, then overall analyte averages over the first period of time may be based on averaging sub-averages even beyond the third sub-average, such as fourth sub-averages (e.g., averages of at least some, and preferably all the third sub-averages), fifth sub-averages (e.g., averages of at least some, and preferably all the fourth sub-averages), etc.

Thus, the overall analyte average 418 may have an advantage that it is less affected by time-varying data and/or by the missing and/or improper data points. Moreover, the sub-averages for each of the sub-periods may likewise be less affected by the missing and/or improper data points contained therein. Furthermore, a user may easily display the overall analyte average 418 for the selected first period of time 402, as well as various overall sub-averages for any other desired sub-period (e.g., 414-414C, 416-416A) within the first period of time 402 as well as plots of the distribution of sub-averages.

For example, the first period of time 402 may relate to a 12-hour period, thus each second sub-average (414-414C) may relate to three hours, and each of the first sub-averages 412 of the time increments 404 may relate to a one hour sub-period. As should be recognized, in an alternate embodiment, each of the time increments 404 may be selected to be other periods of length, such that sub-averages for any desired sub-period may be displayed. For example, one-hour averages may be plotted and displayed. Daytime and nighttime sub-averages may be displayed such as shown in FIG. 6C. An overall daily sub-average may be obtained by averaging 24 hourly increments, for example. An overall weekly sub-average may be provided by averaging seven daily sub-averages. A monthly sub-average may be obtained by averaging the respective weekly sub-averages. Thus, depending on the length of the period of time selected by the user and the amount of analyte readings available, an hourly, daily, daytime, nighttime, weekly, and/or monthly sub-average may be calculated, plotted and/or displayed.

Table 1 below outlines respective analyte concentration averages calculated for various data sets obtained by two prior art methods versus the present invention calculation method and illustrates that the various methods may achieve relatively large differences in the averages calculated thereby.

TABLE 1
Comparative Averages
Calculation Method

| Running | Time-Weighted | Present Invention |
|---|---|---|
| Average | Average | Overall Average |
| (mg/dL) | (mg/dL) | (mg/dL) |
| 160.89 | 176.58 | 177.76 |
| 151.66 | 152.75 | 153.58 |
| 148.91 | 122.67 | 146.06 |
| 251.84 | 182.62 | 259.22 |
| 153.46 | 229.34 | 182.40 |
| 168.80 | 200.34 | 187.58 |

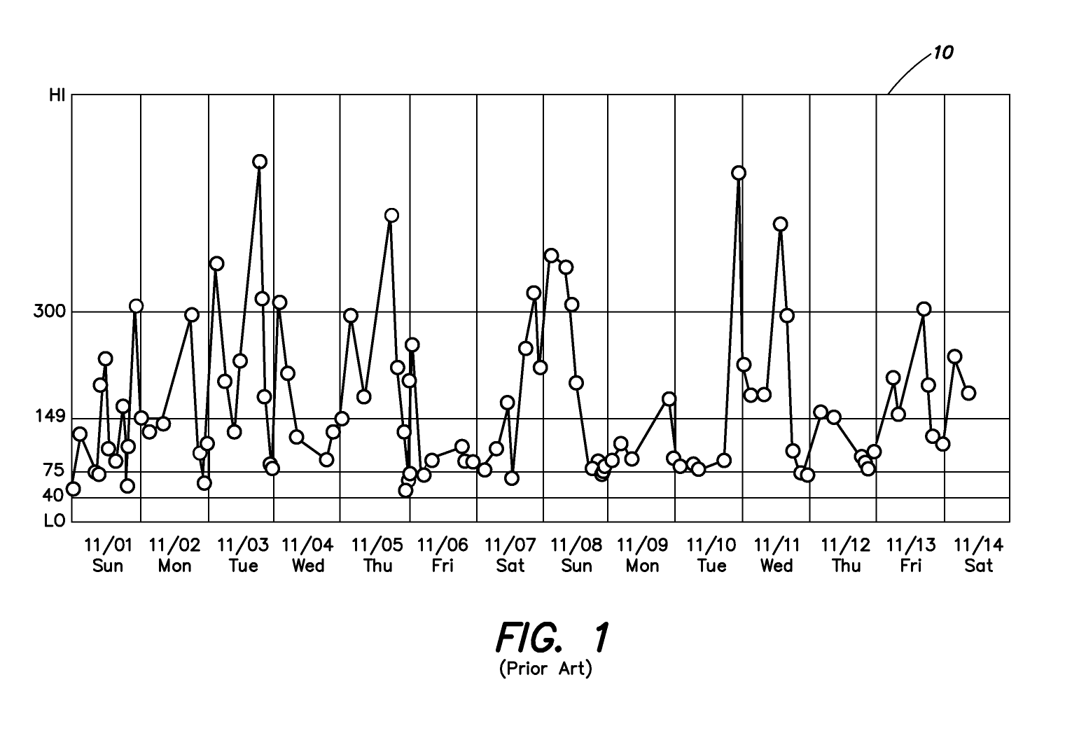

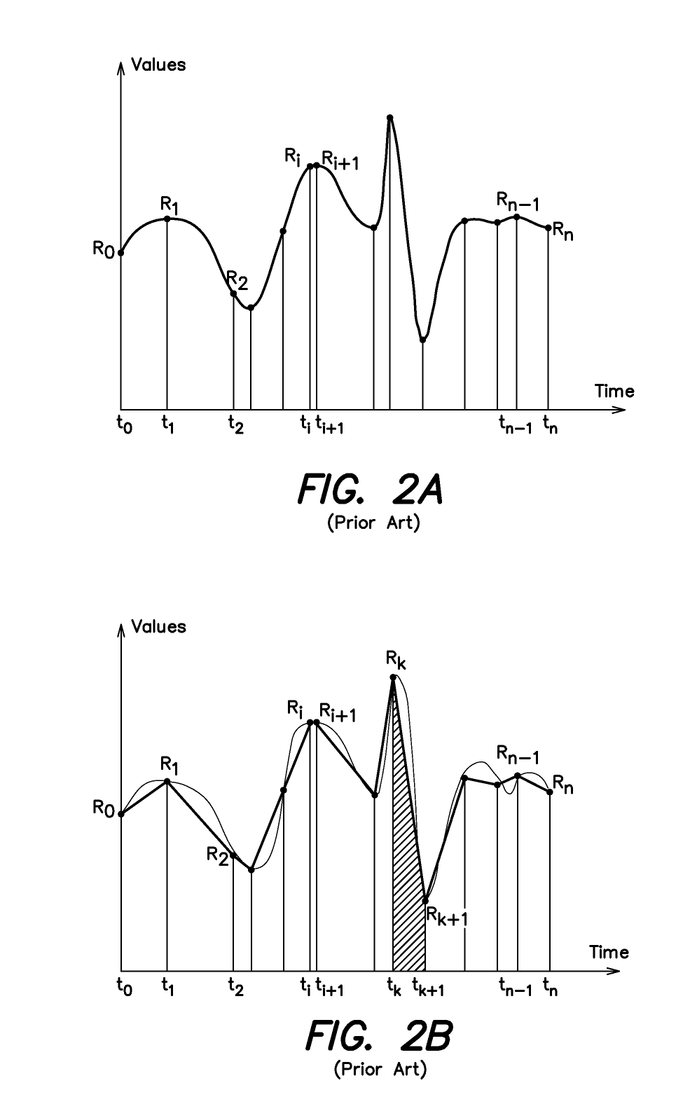

In the prior art running average method, a group of consecutive analyte readings, such as a subgroup of the data shown in FIG. 1, may be averaged over a period of time to determine a running average. As the next reading in time is taken and received, an earliest received reading is dropped from the calculation. Thus, the running average is based upon a set number of data points being averaged within a moving time window. The prior art Time-Weighted Average method, as described in U.S. Patent Application Pub. No. 2007/0010950 involves assigning a time and analyte value to each data point as shown in FIG. 2a, and then providing a trapezoidal approximation for each as shown in FIG. 2b. As can be seen, the method of the present invention results in an overall average value which is sometimes similar and sometimes quite different that the other prior methods. In one advantage, the present invention more accurately accounts for uneven spacing over time of the analyte concentration readings, and in particular, may account for large gaps in the data. In another aspect, the present invention may account for missing, improper and/or otherwise discarded readings. Thus, the present invention may provide a robust calculation method, which may account for such anomalies in the analyte concentration readings.

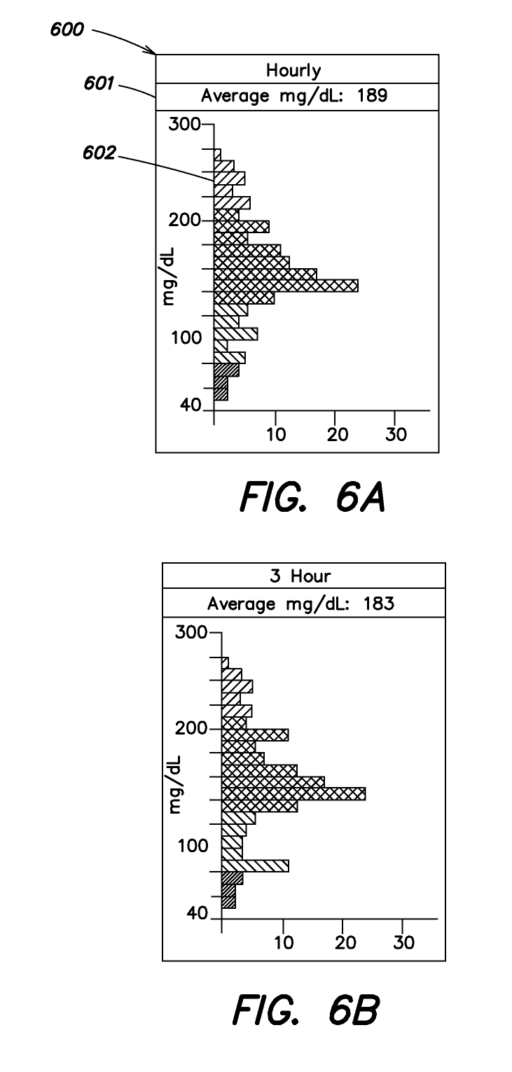

FIG. 5 illustrates an alternative embodiment of the method. This method is similar to the method described with reference to FIG. 4 in that a plurality of analyte concentration readings 501 are stored in memory after having been obtained by a measurement device over a period of time (t). The data may have been downloaded to a host device for processing. Some readings are missing 507 and some have been deemed to be improper 509, as heretofore described. Each analyte reading 501 may have an associated date and time stamp. In this example, the time (t) represents ten days of stored analyte data readings and represents the total amount of data that is available. The user may select any subset of that time for analysis and display. For example, the user may select nine days for the first period of time 502. Other shorter lengths could also be selected. The method may then calculate averages for useful periods of time occurring within the selected first period of time 502. Although not shown, each of the daily increments may be further broken down into suitable length periods (e.g., hourly). Other period lengths may be used. In contrast to the previous embodiment, in this embodiment, the smaller time increments are selected based on preset intervals desired to be displayed (e.g., hourly, daily, daytime (e.g. 6:00 AM to 11:00 PM), nighttime (e.g. 11:00 PM to 6:00 AM), weekly, etc.). Thus, the only sub-averages calculated are those to be displayed.

According to some embodiments of the method, the first sub-averages 512 may be the arithmetic average of the nondiscarded readings (e.g., 3 to 100 readings) from the first increment, where the smaller time increments dividing the first period of time 502 selected are useful preselected increments (e.g., days). As in the previous embodiments, an overall first sub-average over the selected first period of time 502 may be calculated. Second sub-averages 514 may then be obtained by averaging a preset number of adjacent ones (but at least two) of the first sub-averages 512. For example, the second largest useful time increment may be a week, so averages for seven of the smaller daily increments may be calculated, and may be used to calculate the second sub-average 514. In some instances, the first period of time 502 selected by the user may represent less than the total amount of time for which data is stored. For example, selected period of time 502 may be an odd number such that the integer divisions created by the preselected increments by the method result in displayed data being in increments less that the selected first period of time 502. In this instance, some of the sub-averages will be unable to be completed.

For example, the last two days of the selected first period of time 502 are insufficient to calculate another second sub-average. Thus, for the period of interest to the user (e.g., 504), the first sub-averages 512, and a single second sub-average 514 may be calculated. The weekly sub-average may be based on any seven days of the data range selected, such as the first seven, last seven, or seven about the midpoint. As long as sufficient data is present, finer preselected smaller time increments (less than 24 hours) may also be displayed. At least three data points should be available within the increment in order to display a result for that increment. If sufficient data were present and the period of time 504 long enough, then there may be two or more second sub-averages 514 calculated. An overall second sub-average may be calculated and displayed based on the average of the two or more second sub-averages 514. If only one second sub-average 514 is available, then that second sub-average may be displayed for the smaller time increment.

As discussed above, the analyte readings, sub-averages and/or overall analyte sub-averages may be displayed to the user as shown in FIGS. 6A-6C, for example. The overall analyte sub-averages may be displayed for any suitable period, such as an hour, daytime, nighttime, 24 hours, a week, a month, and a year. Similarly, sub-average readings may be displayed for suitable time periods corresponding with events such as breakfast, lunch, and/or dinner. If meal markers (shown as unfilled circles—See FIG. 4) or insulin markers (shown as X-marks—See FIG. 4) are included in the data set, then average readings may be displayed which are associated with these event markers. For example, meal sub-averages for a several hour period after each of the meal events may be calculated displayed as shown in FIG. 6C. An overall meal sub-average over the selected period of time (e.g. 402) may be displayed. Distribution of the meal sub-averages over the selected period of time (e.g. 402) may be displayed.

As shown in FIGS. 6A-6C, statistical data of calculated sub-averages and outliers may be displayed. For example, a distribution of the analyte data 602 obtained over the selected first period of time 502 may be provided. The data groups may be suitably colored to reflect conditions of being within an acceptable range, conditions indicating a high range (e.g., hyperglycemia), and/or conditions indicating a low range (e.g., hypoglycemia). FIGS. 6A-6C illustrate glucose readings for diabetes monitoring, but the present invention may find utility for a broad number of disease conditions or for monitoring other analytes of interest, as mentioned before herein.

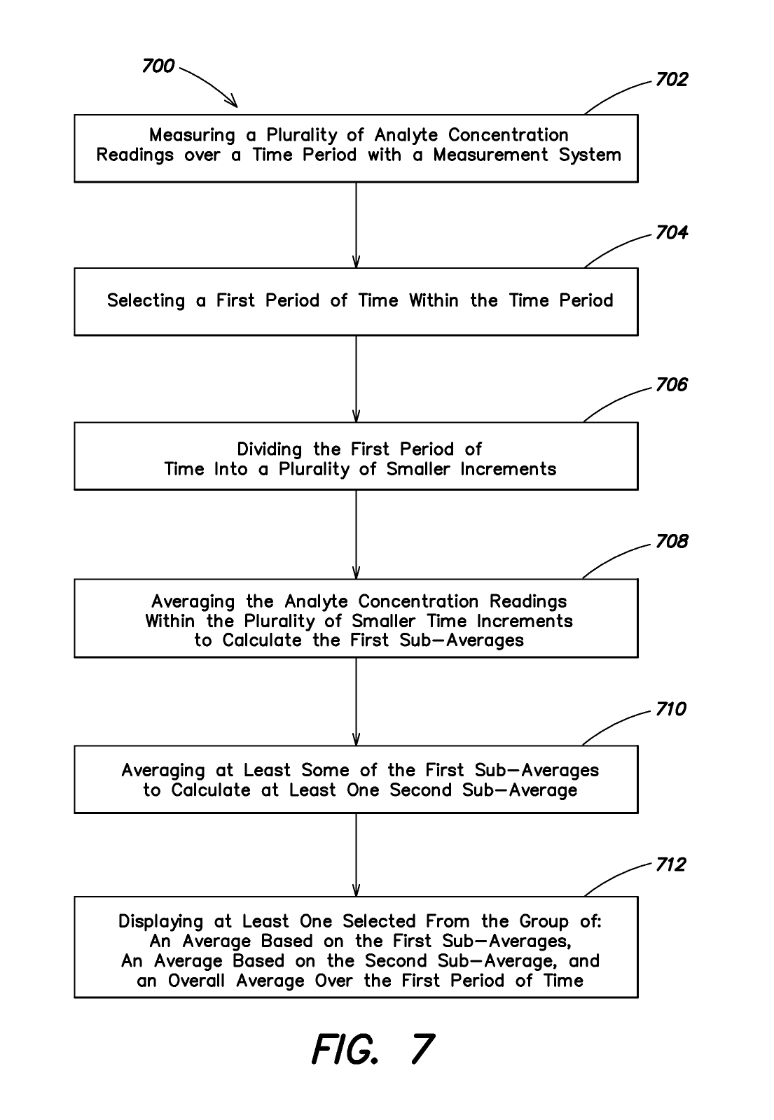

FIG. 7 illustrates a method according to embodiments of the invention. The method 700 may be used to determine one or more average analyte values. According to the method, in 702, a plurality of analyte concentration readings are measured over a time period with an analyte measurement device. Any suitable measurement device may be used, such as those described above. From the time period, a first period of time is selected in 704. The first period of time may be a length of time that a user manually selects, or the first period of time may be a length of time within the time period for which an integer number of the largest increments to be displayed may be evenly divided, or it may be equal to the time period (all the data). Any amount of the data, all or only that for the first period of time, may be downloaded to a host device 310 wherein the calculations according to the invention may take place. Optionally, the calculations may take place within the measurement device 300, assuming sufficient processing power and memory is available. In 706, the first period of time may be divided into a plurality of smaller time increments. The number of increments (m) may be selected as described above. In 708, the analyte concentration readings within the plurality of smaller time increments are averaged to calculate first sub-averages. The minimum number of readings in each increment may be three or more. Averaging calculations may be undertaken for all of the smaller time increments to calculate first sub-averages. From at least two of the first sub-averages, at least one second sub-average may be calculated in 710. As the calculations are completed, at least one average may be displayed in 712. The average displayed may be selected from the group of:
an average based on the first sub-averages,
an average based on the at least one second sub-average, and
an overall analyte average over the first period of time based on the at least one second sub-average.
   In addition, the distribution of any of the calculated sub-averages may be displayed. This may aid the user in determining the extent of the outliers.

While the invention is susceptible to various modifications and alternative forms, specific embodiments and methods thereof have been shown by way of example in the drawings and are described in detail herein. It should be understood, however, that it is not intended to limit the invention to the particular systems or methods disclosed, but, to the contrary, the intention is to cover all modifications, equivalents and alternatives falling within the spirit and scope of the invention.

What is claimed is:

1. A method to determine an average analyte value, comprising:
measuring a plurality of analyte concentration readings over a time period with an analyte measurement device;
selecting a first period of time within the time period;
dividing the first period of time into a plurality of smaller time increments each including at least three analyte concentration readings;
averaging the analyte concentration readings within the plurality of smaller time increments to calculate first sub-averages;
averaging two or more of the first sub-averages to calculate at least one second sub-average; and
displaying at least one average selected from the group of:
an average based on the first sub-averages,
an average based on the at least one second sub-average, and
an overall analyte average over the first period of time based on the at least one second sub-average.

2. The method of claim 1 further comprising downloading the plurality of analyte concentration readings to a host device.

3. The method of claim 1 wherein each of the plurality of smaller time increments are substantially equal length time increments.

4. The method of claim 1 further comprising not using in the calculation of the first sub-averages any analyte concentration reading which is missing.

5. The method of claim 1 further comprising not using in the calculation of the first sub-averages any analyte concentration reading which is determined to be an improper analyte reading.

6. The method of claim 5 wherein the improper analyte reading is determined to be improper by comparing the improper analyte reading to at least one characteristic of at least one previously-obtained analyte reading.

7. The method of claim 1 wherein the at least one second sub-average is over a sub-increment of time smaller than the first period of time.

8. The method of claim 1 wherein the analyte concentration readings are unequally spaced in time over the first period of time.

9. The method of claim 1 wherein the analyte measurement system is one system selected from a group consisting of a BGM, a CGM, CGM/Pump combination.

10. The method of claim 1 wherein the smaller time increment is one time increment selected from the group consisting of 15 minutes, a half hour, an hour, 24 hours, a week, and a month.

11. The method of claim 1 wherein the analyte concentration readings comprise glucose concentration readings.

12. The method of claim 1 wherein the overall analyte average is based at least in part on an arithmetic average of the at least one second sub-average and at least one other second sub-average.

13. The method of claim 1 wherein plurality of smaller time increments include between 3 and 100 analyte concentration readings.

14. The method of claim 1 wherein all second sub-averages over the first period of time are averaged.

15. The method of claim 1 wherein the at least one second sub-average is averaged with at least one other second sub-average to produce a third sub-average.

16. The method of claim 1 wherein all second sub-averages are averaged to produce an overall second sub-average.

17. The method of claim 1 wherein the overall analyte average over the first period of time is based at least in part on an average of third sub-averages.

18. The method of claim 17 wherein the overall analyte average over the first period of time is based on averaging sub-averages beyond the third sub-average.

19. The method of claim 1 wherein at least some of the plurality of analyte concentration readings within the plurality of smaller time increments are discarded.

20. The method of claim 1 wherein a number (m) of the plurality of smaller time increments is selected such that each of the plurality of smaller time increments includes at least a number (k) of the analyte concentration readings wherein m>5 and k>5.

21. A method to determine an average analyte value, comprising:
measuring a plurality of analyte concentration readings with an analyte measurement system over a time period;
downloading the plurality of analyte concentration readings to a host device;
selecting a first period of time within the time period,
dividing the first period of time into a plurality of smaller time increments of substantially equal lengths;
averaging the analyte concentration readings for each of the smaller time increments to determine first sub-averages for each of the smaller time increments;
averaging the first sub-averages to arrive at a plurality of second sub-averages;
averaging the second sub-averages to arrive at at least one third sub-average; and
displaying on the host device at least one average selected from the group of:
the first sub-averages,
the second sub-averages,
the at least one third sub-average, and
an overall analyte average over the first period of time.

22. A system adapted to calculate an average analyte value, comprising:
a measurement system adapted to obtain a plurality of analyte concentration readings;
a host device adapted to receive at least some of the plurality of analyte concentration readings, the host device including:
a processor adapted to calculate an analyte concentration average over a selected first period of time wherein a plurality of analyte readings over the first period of time are stored in memory and the processor calculates an analyte concentration average based on:
dividing the first period of time into a plurality of smaller time increments, each smaller time increment including at least three analyte concentration readings,
averaging the analyte concentration readings for each of the smaller time increments to determine a first sub-average for each of the smaller time increments, and
averaging at least some of the first sub-averages to arrive at at least one second sub-average.
